(19) United States
(12) Patent Application Publication
(10) Pub. No.: US 2024/0307482 A1
(43) Pub. Date: Sep. 19, 2024

(54) TOPICAL COMPOSITIONS

(71) Applicant: Illustris Pharmaceuticals, Inc.

(72) Inventor: Siak Khim TAN, Singapore (SG)

(21) Appl. No.: 18/735,024

(22) Filed: Jun. 5, 2024

Related U.S. Application Data
(63) Continuation of application No. 17/869,665, filed on Jul. 20, 2022, now Pat. No. 12,036,263.
Application No. 17/869,665 is a continuation of application No. 16/920,460, filed on Jul. 3, 2020, now abandoned.
(60) Provisional application No. 62/870,154, filed on Jul. 3, 2019.

(51) Int. Cl. A61K 38/08; A61K 9/00; A61K 9/06; A61K 31/728; A61K 38/05; A61K 38/06; A61K 38/07; A61K 38/39

(52) U.S. Cl. CPC A61K 38/08; A61K 9/0014; A61K 9/06; A61K 31/728; A61K 38/05; A61K 38/06; A61K 38/07; A61K 38/39

(57) ABSTRACT

Compositions for dermal delivery of a topical solution and methods for using such compositions are described herein. The compositions may contain extracellular matrix components and therapeutic peptides.

### CROSS-REFERENCE TO RELATED APPLICATIONS

This application is a continuation of U.S. application Ser. No. 17/869,665, filed Jul. 20, 2022, allowed, which is a continuation of U.S. application Ser. No. 16/920,460, filed Jul. 3, 2020, abandoned, which claims the benefit of priority to U.S. Provisional Patent Application No. 62/870, 154, filed Jul. 3, 2019, each of which is incorporated herein by reference in its entirety.

### SEQUENCE LISTING

The instant application contains a Sequence Listing which has been filed electronically in XML format and is hereby incorporated by reference in its entirety. Said XML copy, created on Jun. 4, 2024, is named 090471-0103-8003.US02_SEQ.xml and is 9,000 bytes in size.

### BRIEF SUMMARY

Various embodiments are directed to compositions including an extracellular matrix component or fragment thereof having a concentration of about 0.1 wt. % to about 5 wt. % based on the total weight composition and a therapeutic peptide having a concentration of about 0.1 wt. % to about 25 wt. % based on the total weight composition. In some embodiments, the extracellular matrix component or fragment thereof may be hyaluronic acid, collagen, fibronectin, elastin, lectin, and the like and combinations thereof, and in some embodiments, collagen may be collagen type I, collagen type II, collagen type III, collagen type IV, collagen type V, fibrillary collagen, non-fibrillary collagen, and combinations thereof. In some embodiments, the therapeutic peptide may be palmitoyl oligopeptide, palmitoyl tripeptide-1, palmitoyl tetrapeptide-7, acetyl hexapeptide-3, and the like and combinations thereof. In some embodiments, the composition may include about 1 mg to about 1000 mg of the extracellular matrix component or a fragment thereof, and in some embodiments, the composition may include about 1 mg to about 1000 mg therapeutic peptide. In some embodiments, the concentration of therapeutic peptide may be about 5 wt. % to 20 wt. %, and in some embodiments, the therapeutic peptide may be dipeptides, tripeptides, tetrapeptides, pentapeptides, hexapeptides, dipeptide derivatives, tripeptides derivatives, tetrapeptides derivatives, pentapeptides derivatives, hexapeptides derivatives, and the like and combinations thereof.

The composition may include one or more pharmaceutical additives such as, for example, diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, or combinations thereof, and in some embodiments, the composition may include one or more cosmetic additives such as vitamins, cosmetic peptides, oil control agents, other skin care agents, and hydrating compositions. Such composition may be formulated as a liquid, cream, ointment, gel, or the like.

Additional embodiments are directed to methods for treating skin by topically administering the compositions described above.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey the scope to those skilled in the art.

Where a range of values is provided, it is intended that each intervening value between the upper and lower limit of that range and any other stated or intervening value in that stated range is encompassed within the disclosure. For example, if a range of I μm to 8 μm is stated, it is intended that 2 μm, 3 μm, 4 μm, 5 μm, 6 μm, and 7 μmare also explicitly disclosed, as well as the range of values greater than or equal to I μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The compositions of the present disclosure can comprise, consist essentially of, or consist of, the components disclosed.

All percentages, parts and ratios are based upon the total weight of the topical compositions and all measurements made are at about 25° C., unless otherwise specified.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to directly administering a compound (also referred to as an agent of interest).

As used herein, the term “cosmetic” or “cosmetics” are substances or products used to enhance or alter the appearance of the face or body often called “make-up” or “makeup.” The terms “skin care composition” or “skin care product” is used to describe compositions that clean or treat skin, and include, for example, cleansers, lotions, soaps, balms, foams, and the like.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum comeum or stratum spinosum.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopcia for use in mammals (e.g, animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient,” “subject,” and “user” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition in a subject relative to a subject not receiving the compound or composition. This can include reversing, reducing, or arresting the symptoms, clinical signs, and underlying pathology of a condition in a manner to improve or stabilize a subject's condition.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments of the invention are directed to topical compositions containing a combination of an extracellular matrix molecule and a therapeutic peptide, and methods for treating disorders of the epidermis or skin using such topical compositions. The compositions of embodiments may create an optimal environment for the body's innate processes of repair and renewal and ensure absorption and delivery of essential ingredients.

“Topical administration” is used in its conventional sense to mean delivery of a composition to the skin or mucosa, as in, for example, the treatment of various skin disorders. Topical administration, in contrast to transdermal administration, provides a local rather than a systemic effect. However, unless otherwise stated or implied, the terms “topical product administration” and “transdermal product administration” are used interchangeably.

In certain embodiments, the extracellular matrix molecule may be a fragment of an extracellular matrix component or a receptor associated with the extracellular matrix. For example, in some embodiments, the extracellular matrix molecule may be hyaluronic acid, elastin, collagen, fibronectin, lectin, and the like and combinations thereof.

In various embodiments, the extracellular matrix molecule may have an average molecular weight of less than 100,000 Daltons (“Da”). In particular embodiments, the extracellular matrix molecule may have an average molecular weight of about 2,000 Da to about 60,000, about 2,000 Da to about 40,000 Da, or about 5,000 Da to about 40,000 Da.

In general, the amount of extracellular matrix molecules present in the compositions of various embodiments may be from about 0.1 wt. % to about 10 wt. %, and in particular embodiments, the amount of extracellular matrix molecules in such compositions may be from about 0.1 wt. % to about 2.0 wt. %, about 0.25 wt. % to about 3.0 wt. %, about 0.5 wt. % to about 5.0 wt. %, about 0.75 wt. % to about 7.5 wt. %, or any range or individual concentration encompassing these example ranges. In some embodiments, the amount of extracellular matrix molecules in a composition may be about 1 mg to about 1000 mg, about 1 mg to about 900 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 10 mg to about 1000 mg, about 50 mg to about 1000 mg, about 100 mg to about 1000 mg, about 200 mg to about 1000 mg, about 300 mg to about 1000 mg, about 400 mg to about 1000 mg, about 500 mg to about 1000 mg, about 10 mg to about 500 mg, about 50 mg to about 500 mg, about 100 mg to about 500 mg, about 10 mg to about 300 mg, about 50 mg to about 300 mg, from about 100 mg to about 300 mg, about 10 mg to about 150 mg, about 50 mg to about 150 mg, about 60 mg to about 120 mg, about 50 mg to about 120 mg or a range between any two of these values.

In particular embodiments, the extracellular matrix molecule may be hyaluronic acid. Hyaluronic acid is known to interact with, for example, CD44, receptor for hyaluronic acid-mediated motility (RHAMM), and intercellular adhesion molecule-I (ICAM-1). CD44 is widely distributed throughout the body and mediates cell interaction with hyaluronic acid. ICAM-1 is a metabolic cell surface receptor for hyaluronic acid, and binding of hyaluronic acid to ICAM-1 may contribute to the control of ICAM-1-mediated inflammatory activation. Hyaluronic acid is polymer of disaccharides. Without wishing to be bound by theory, low molecular weight fragments of hyaluronic acid may disrupt cell-cell and cell-scaffold attachments by interrupting intercellular interactions and/or by triggering cellular injury response, which may disrupt intercellular interactions between cells that do not directly contact the hyaluronic acid molecule.

In some embodiments, the extracellular matrix molecule may be collagen. Collagen can be isolated in various forms and from a number of sources. Example collagens include collagen type I, collagen type II, collagen type III, collagen type IV, or collagen type V. The collagen can also be fibrillary collagen or non-fibrillar collagen. Low molecular weight collagens can be made, for example, by hydrolysis, and like hyaluronic acid, low molecular weight collagen may disrupt cell-cell and cell-scaffold attachments by interrupting intercellular interactions and/or by triggering cellular injury response, which may disrupt intercellular interactions between cells deeper in the tissue.

In certain embodiments, the extracellular matrix molecule may be fibronectin. Fibronectin is a protein dimer, consisting of two nearly identical monomers linked by a pair of disulfide bonds. Fibronectin binds to membrane-spanning receptor proteins called integrins and extracellular matrix components such as collagen, fibrin, and heparin sulfate proteoglycans. Like hyaluronic acid and collagen, fibronectin fragments may disrupt cell-cell and cell-scaffold attachments by interrupting intercellular interactions and/or by triggering cellular injury response, which may disrupt intercellular interactions between cells deeper in the tissue.

In some embodiments, the extracellular matrix molecule may be elastin. Elastin is a protein found in connective tissue and allows many tissues in the body to resume their shape after stretching or contracting. Like hyaluronic acid, collagen, and fibronectin, elastin fragments may disrupt cell-cell and cell-scaffold attachments by interrupting intercellular interactions and/or by triggering cellular injury response, which may disrupt intercellular interactions between cells deeper in the tissue.

The therapeutic peptides of various embodiments include, without limitation, the di-, tri-, terra-, penta- and hexapeptides and their derivatives. The term “peptide” refers to peptides containing 10 amino acids or less, their derivatives, isomers and complexes with other species such as a metallic ion (e.g. copper, zinc, manganese, magnesium, and others). The term “peptides” refers to both natural peptides and synthetic peptides. It also refers to compositions that contain peptides that are found in nature, isolated from natural sources, synthesized, are commercially available, and the like.

Dipeptides encompassed by the invention include, but are not limited to, carnosine (beta-AH), YR, VW, NF, DF, KT, KC, CK, KP, KK, TT, and the like and combinations thereof. Tripeptides include, but are not limited to, RKR, HGG, GKH, GGH, GHG, KFK, KPK, KMOK, KM02K, KAvaK, and the like and combinations thereof. Tetrapeptides include, but are not limited to, RSRK, KTFK, and the like and combinations thereof. Non-limiting examples of pentapeptide include KTTKS (SEQ ID NO: 1) and the like and of hexapeptides the GKTTKS (SEQ ID NO: 2), VGVAPG (SEQ ID NO: 3), and the like and combinations thereof.

Other suitable therapeutic peptides include, but are not limited to, lipophilic derivatives of peptides, including, for example, palmitoyl derivatives and metal complexes such as, for example, a copper complex of the tripeptide HGG. Dipeptide derivatives include Pal-beta-AH, Ac-YR-hexadecylester, Pal-KT, Pal-RT, and the like and combinations thereof. Tripeptide derivatives include the copper derivative of HGG, Lipospondin (Ela-KFK) and its analogs, Ac-RKR-NH2 (Peptide CK+), Pal-KM02K, and the like and combinations thereof.

Tetrapeptide derivatives include Ela-KTFK, and pentapeptide derivatives include, Pal-KTTKS (SEQ ID NO: 4), Pal-YGGFX (SEQ ID NO: 5) with X being Met(M) or Leu(L) and the like and combinations thereof. Hexapeptide derivatives include Pal-VGVAPG (SEQ ID NO: 6), Pal-GKTTKS (SEQ ID NO: 7), and the like and combinations and derivatives thereof. In certain embodiments, the compositions may include a combination of various dipeptides, tripeptides, tetrapeptides, pentapeptides, hexapeptides, dipeptide derivatives, tripeptides derivatives, tetrapeptides derivatives, pentapeptides derivatives, hexapeptides derivatives, and the like.

The following commercially available therapeutic peptides can be mentioned as well as additional active ingredients: Vialox™, Syn-ake™ or Syn-Coll™ (Pentapharm), Hydroxyprolisilane CN™ (Exsymol), Argireline™, Leuphasyl™, Aldenine™, Trylgen™, Eyeseryl™, Serilesine™ or Decorinyl™ (Lipotec), Collaxyl™ or Quintescine™ (Vincience), BONT-L-Peptide™ (Infinitec Activos), Cytokinol™LS (Laboratoires Serobiologiques/Cognis), Kollaren™, IP2000™ or Meliprene™ (Institut Europeen de Biologie Cellulaire), Neutrazen™ (Innovations), ECM-Protect™M (Atrium Innovations), Timp-Peptide™, ECM Moduline™ (Infinitec Activos). Palmitoyl oligopeptide (e.g., palmitoyltripeptide) and/or palmitoyltetrapeptide-7, and derivatives thereof, commonly known as MATRIXYL™3000 (manufactured by Sederma, SA), is a blend of two or more peptide complexes. Palmitoyloligopeptide (e.g., palmitoyltripeptide), as set forth above, is a peptide comprised of 3 amino acids: Glycine Histidine-Lysine (N-palmitoyl-Gly-His-Lys). In some embodiments, therapeutic peptides may include palmitoyl tetrapeptide-7. This therapeutic peptide may be composed of four amino acids plus fatty acid palmitic acid to enhance stability and penetration into the skin. Therapeutic peptides have different functions on skin, and this particular peptide, on its own or as part of Matrixyl 3000, helps to replenish the skin's surface and calm visible signs of sensitization.

In some embodiments, therapeutic peptides may include acetyl hexapeptide-3, otherwise known as argireline. This therapeutic peptide may bind to the SNARE-receptor, blocking the production of neurotransmitter which then reduces facial muscle contraction. It has been used as a replacement for BOTOX®. Formulations with 10% concentration acetyl hexapeptide have been shown to reduce the depth of wrinkles up to 30% after 30 days of trial use. The skincare composition may include hexapeptide complexes having hexapeptide concentrations ranging from approximately 0.00001 to approximately 15% w/w, preferably from 0.01% w/w to 10% w/w, and more preferably from 0.1 to 5%.

Particular examples of compositions encompassed by the invention include compositions containing about 0.1 wt. % to about 2.0 wt. % extracellular matrix molecule having an average molecular weight of about 2,000 Da to about 60,000 Da, and a peptide such as palmitoyl oligopeptide (palmitoyl tripeptide-1, palmitoyl tetrapeptide-7, acetyl hexapeptide-3, and peptides of similar size and combinations thereof.

In some embodiments, the compositions described above may further include one or more pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and the like and combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of embodiments.

In some embodiments, the compositions described above may be formulated as a liquid. Liquid dosage forms for topical administration may include diluents such as, for example, alcohols, glycols, oils, water, and the like. Such compositions may also include wetting agents or emulsifiers. In some embodiments, the compositions of embodiments may be formulated as oil-in-water or water-in-oil emulsion. A cream can be a water-in-oil (w/o) emulsion in which an aqueous phase is dispersed in an oil phase, or an oil-in-water (o/w) emulsion in which an oil is dispersed within an aqueous base. An ointment generally refers to a more viscous oil-in-water cream. Traditional ointment bases (i.e. carrier) include hydrocarbons (petrolatum, beeswax, etc.) vegetable oils, fatty alcohols (cholesterol, lanolin, wool alcohol, stearyl alcohol, etc.) or silicone. Insoluble solids such as starch, zinc oxide, calcium carbonate, or talc can also be used in ointments and creams. Gel forms of the compositions described above can be formed by the entrapment of large amounts of aqueous or aqueous-alcoholic liquids in a network of polymers or of colloidal solid particles. Such polymers or colloids (gelling or thickening agents) are typically present at concentrations of less than 10% w/w and include carboxymethyl cellulose, hydroxypropylmethyl cellulose, hydroxyethyl cellulose, methyl cellulose, sodium alginate, alginic acid, pectin, tragacanth, carrageen, agar, clays, aluminum silicate, carbomers, and the like.

Emollient or lubricating vehicles that help hydrate the skin can also be used. Examples of suitable bases or vehicles for preparing hydrating compositions for use with human skin are petrolatum, petrolatum plus volatile silicones, lanolin, cold cream (USP), and hydrophilic ointment (USP).

In certain embodiments, the compositions of various embodiments may be formulated for improving appearance of skin and may additionally include additives such as vitamins, cosmetic peptides, oil control agents, and other skin care agents.

Vitamins include, for example, vitamin D, vitamin K, vitamin B (including niacinamide, nicotinic acid, C1_18 nicotinic acid esters, and nicotinyl alcohol; B6 compounds, such as pyridoxine; and BS compounds, such as panthenol, or “pro-BS”), vitamin A (including retinoids such as retinyl propionate, carotenoids, and other compounds), vitamin E (including tocopherol sorbate, tocopherol acetate, other esters of tocopherol), vitamin C (including ascorbyl esters of fatty acids, and ascorbic acid derivatives, for example, ascorbyl glucoside, magnesium ascorbyl phosphate, sodium ascorbyl phosphate, and ascorbyl sorbate), and all natural and/or synthetic analogs thereof, and combinations thereof. In various embodiments, the compositions may include about 0.0001 wt. % to about 50 wt. %, about 0.001 wt. % to about 10 wt. %, about 0.01 wt. % to about 5 wt. %, or about 0.1 wt. % to about 1 wt. %, or any individual concentration or range of each vitamin contained in the composition.

Peptides include di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (MATRIXYL®) palmitoyl-glycine-glutamine-proline-arginine (RIGIN®), these three being available from Sederma, France, and Cu-histidine-glycine-glycine (Cu-HGG, also known as IAMIN®), and naturally occurring and synthesized derivatives thereof, and combinations thereof. In various embodiments, the compositions may include about 1×10−7 wt. % to about 20 wt. %, about 1×1o−6 wt. % to about 10 wt. %, and about 1×10−5 wt. % to about 5 wt. %, or any individual concentration or range of each peptide contained in the composition.

Oil control agents include compounds useful for regulating the production of skin oil, or sebum, and for improving the appearance of oily skin. Examples of oil control agents include, for example, salicylic acid, dehydroacetic acid, benzoyl peroxide, vitamin B3 (for example, niacinamide), and the like, their isomers, esters, salts and derivatives, and mixtures thereof. The compositions of such embodiments may include about 0.0001 wt. % to about 15 wt. %, about 0.01 wt. % to about 10 wt. %, about O.1 wt. % to about 5 wt. %, and about O.2 wt. % to about 2 wt. %, or any individual concentration or range of each oil control agent contained in the composition.

Other skin care agents include retinal, steroids, sunblock, salicylate, minocycline, antifungals, peptides, antibodies, lidocaine, and the like and combinations thereof. In some embodiments, other skin care agents include N-acyl amino acid compounds including, for example, N-acyl phenylalanine, N-acyl tyrosine, and the like, their isomers, including their D and L isomers, salts, derivatives, and mixtures thereof. An example of a suitable N-acyl amino acid is N-undecylenoyl-L-phenylalanine is commercially available under the tradename SEPIWHITE®. Further skin care agents are disclosed in U.S. Publication No. 2007/0020220A1, wherein the components/ingredients are incorporated herein by reference in their entirety.

Some embodiments may include the step of administering the composition to a surface tissue. For example, such methods may include the step of applying a composition or formulation such as those described above including an extracellular matrix molecule and a peptide to a surface tissue of a subject.

As indicated above, a “surface tissue” includes any surface tissue such as, but not limited to, skin surfaces of the face, hands, neck, and the like. The methods of such embodiments may include a variety of additional steps including, for example, cleaning the surface tissue at the site of applying and the like. In such embodiments, the composition can be applied to the surface tissue one or more times each day, and applying can be carried out for a period of at least 1 month, 2 months, 3 months, 4 months, 6 months, 8 months or 12 months.

The methods of embodiments may be used for administration of various cosmetic therapies for improving, for example, skin thickness, elasticity, resiliency, smoothness, tone, texture, brightness, clarity, contour, firmness, tautness, suppleness, discoloration, skin lesions, and the like and combinations thereof.

It is claimed:

1. A composition, comprising:
an extracellular matrix component and a therapeutic peptide,
wherein the extracellular matrix component consists of an extracellular matrix component with an average molecular weight of less than about 100,000 Daltons; and
wherein the therapeutic peptide is at a concentration of about 0.1 wt. % to about 25 wt. % based on the total weight of the composition.

2. The composition of claim 1, wherein the extracellular matrix component is a component or a fragment thereof selected from the group consisting of hyaluronic acid, collagen, fibronectin, elastin, and lectin.

3. The composition of claim 2, wherein collagen is selected from the group consisting of collagen type I, collagen type II, collagen type III, collagen type IV, collagen type V, fibrillary collagen, non-fibrillary collagen, and a combination thereof.

4. The composition of claim 1, wherein the therapeutic peptide is a peptide selected from the group consisting of palmitoyl oligopeptide, palmitoyl tripeptide-1, palmitoyl tetrapeptide-7, acetyl hexapeptide-3, and combinations thereof.

5. The composition of claim 1, wherein the extracellular matrix component is at a concentration of about 0.1 wt. % to about 5 wt. % based on the total weight of the composition.

6. The composition of claim 1, wherein the extracellular matrix component consists of an extracellular matrix component with an average molecular weight of between about 2,000-60,000 Daltons.

7. The composition of claim 1, wherein the concentration of therapeutic peptide is about 5 wt. % to 20 wt. %.

8. The composition of claim 1, further comprising one or more pharmaceutical additives selected from the group consisting of diluents, fillers, disintegrants, binders, lubricants, surfactants, hydrophobic vehicles, water soluble vehicles, emulsifiers, buffers, humectants, moisturizers, solubilizers, preservatives, colorants, plasticizers, carriers, excipients, and a combination thereof.

9. The composition of claim 1, wherein the therapeutic peptide is selected from the group consisting of dipeptides, tripeptides, tetrapeptides, pentapeptides, hexapeptides and a combination thereof.

10. The composition of claim 9, wherein the therapeutic peptide is selected from the group consisting of acetyl hexapeptide-3 and Cu-histidine-glycine-glycine.

11. The composition of claim 9, wherein the therapeutic peptide is a palmitoyl oligopeptide.

12. The composition of claim 11, wherein the therapeutic peptide is selected from the group consisting of palmitoyl tripeptide-1, palmitoyl tetrapeptide-7, palmitoyl-lysine-threonine, palmitoyl-lysine-threonine-threonine-lysine-serine, N-palmitoyl-glycine-histidine-lysine, palmitoyl-glycine-glutamine-proline-arginine, palmitoyl-VGVAPG, palmitoyl-GKTTKS, and palmitoyl-YGGFX, where X is Met(M) or Leu(L).

13. The composition of claim 1, further comprising a vitamin selected from the group consisting of vitamin D, vitamin K, vitamin B, vitamin A, vitamin E, and vitamin C, or a combination thereof.

14. The composition of claim 1, further comprising an oil control agent selected from the group consisting of salicylic acid, dehydroacetic acid, benzoyl peroxide, and vitamin B3, or a mixture thereof.

15. The composition of claim 1, further comprising a skin care agent selected from the group consisting of retinal, salicylate, minocycline, and lidocaine.

16. The composition of claim 1, further comprising a lubricating vehicle selected from the group consisting of petrolatum, petrolatum plus volatile silicone, lanolin, cold cream, and hydrophilic ointment.

17. A method for enhancing or altering appearance of skin, comprising:
topically administering a composition comprising an extracellular matrix component and a therapeutic peptide,
wherein the extracellular matrix component consists of an extracellular matrix component with an average molecular weight of less than about 100,000 Daltons, and wherein the therapeutic peptide is at a concentration of about 0.1 wt. % to about 25 wt. % based on the total weight of the composition.

18. The method of claim 17, wherein the extracellular matrix component is a component or a fragment thereof is selected from the group consisting of hyaluronic acid, collagen, fibronectin, elastin, and lectin.

19. The method of claim 17, wherein the extracellular matrix component is at a concentration of about 0.1 wt. % to about 5 wt. % based on the total weight of the composition.

20. The method of claim 17, wherein the therapeutic peptide is selected from the group consisting of dipeptides, tripeptides, tetrapeptides, pentapeptides, hexapeptides and a combination thereof.


SEQUENCE LISTING (7 sequences)

SEQ ID NO: 1 - protein, 5 aa, synthetic construct
KTTKS

SEQ ID NO: 2 - protein, 6 aa, synthetic construct
GKTTKS

SEQ ID NO: 3 - protein, 6 aa, synthetic construct
VGVAPG

SEQ ID NO: 4 - protein, 5 aa, synthetic construct
  MOD_RES 1: note = modified with palmitoyl
KTTKS

SEQ ID NO: 5 - protein, 5 aa, synthetic construct
  MOD_RES 1: note = modified with palmitoyl
  VARIANT 5: note = X is M or L
YGGFX

SEQ ID NO: 6 - protein, 6 aa, synthetic construct
  MOD_RES 1: note = modified with palmitoyl
VGVAPG

SEQ ID NO: 7 - protein, 6 aa, synthetic construct
  MOD_RES 1: note = modified with palmitoyl
GKTTKS